(19) United States
(12) Patent Application Publication
(10) Pub. No.: US 2015/0031046 A1
(43) Pub. Date: Jan. 29, 2015

(54) Bioanalytical Reagent used in Heterogeneous Phase and Usage Method Thereof

(71) Applicant: Lijun Dai, Changzhou (CN)

(72) Inventor: Lijun Dai, Changzhou (CN)

(21) Appl. No.: 14/119,527

(22) PCT Filed: May 24, 2011
(86) PCT No.: PCT/CN2011/074601

§ 371 Date: Aug. 29, 2014

(30) Foreign Application Priority Data
May 26, 2011 (ES) P201130865

(51) Int. Cl. G01N 33/543; G01N 33/58

(52) U.S. Cl. CPC G01N 33/54346; G01N 33/54353; G01N 33/54306; G01N 33/582
USPC 435/7.1; 530/391.5

(57) ABSTRACT

A bioanalytical reagent used in a heterogeneous phase and a usage method thereof are provided to increase the signal intensity from the bioanalytical reagent. The bioanalytical reagent includes a target detector and a signal generator. The signal generator is represented by the following formula: (p1) _{y}-(a trigger)_{z}-(LS) _{N}-a degradable polymer coupled with a contrast agent in latent state via covalent bonds-p₂, wherein p1 is a protecting group for the end group of the trigger, LS is a self-immolative linker or spacer, p2 is a protecting group for the end group of the degradable polymer, y is 0 or 1, and Z, N are non-negative integer. The signal generator is in a latent state during wash and separation procedures, until an inducing matter or a condition, which excites the contrast agent from a latent state to an active state, is added or applied into the analysis system after the separation procedure so as to degrade directly the polymer or to stimulate the triggers in sequence before degrading the polymer. As a result, the detection signal is excited or its intensity is remarkably increased. The bioanalytical reagent used in a heterogeneous phase has high sensitivity and good stability. The bioanalytical reagent used in a heterogeneous phase is employed simply and exactly.

### TECHNICAL FIELD

The invention belongs to the field of biological testing, in particular to bioanalytical reagent used in heterogeneous phase and usage method thereof.

### BACKGROUND OF THE INVENTION

In biological detection analysis process, according to the differences in analysis process, can be divided into the homogeneous phase and heterogeneous phase two categories. For example, the antigen in the specimen to be detected react with the antibody in the bioanalytical reagent, and form the antigen-antibody complex. Thus, The reagent is divided to reagent (B) which combine the antigen and reagent (F) which do not combine the antigen, or named as combined (B) and free (F); according to the determination of one of the two, the content of the antigen in the specimen to be detected can be calculated. In the general case, it is necessary to separate the combined (B) and free (F) before proceed the measurement, this is heterogeneous phase. In special case, B and F have different characteristics, can be measured without separation, this is homogeneous phase. Homogeneous phase and heterogeneous phase have characteristics respectively in the immunoassay automation design. Most of the labeled immunoassay are belong to heterogeneous phase. The separation method most used for separating the B and F is washing the solid phase carrier.

As shown in FIG. 1, the existing heterophasic bioanalytical reagent fix the target (A) to be detected directly or through the adapter (such as B, the target detector 2) to a solid surface; the detector (target detector 1) in the heterophasic biological analysis which can combine the target directly or indirectly through the adapter is fixed, and then, wash away the uncombined heterophasic biological analytical reagent; finally, The signal generating unit in the signal generator send signals under the action of the external signal source, calculate the content of the target based on the detected signal intensity.

Most of the existing signal generator is a contrast agent, such as organic fluorescent dyes, including fluorescein, rhodamine, and so on. The organic fluorescent dye has the higher efficiency of fluorescence, the longer excitation wavelength, but it is easy to photobleaching, the fluorescence self-quenching effect is serious in the coupling with the biomolecules. Other contrast agents used usually also have the similar problem, the interactions between the single molecule of the contrast reagent, result in the emitted signal intensity reduced.

### SUMMARY OF THE INVENTION

In view of this, the present invention is to overcome the defect of the interactions between the single molecule of the contrast agents existing in the prior art, resulting in the signal intensity reduced. The invention provide a bioanalytical reagent used in heterogeneous phase and usage method thereof. The individual molecules of the contrast reagent in the said heterophasic bioanalytical reagent do not affect each other, it is possible to send a strong signal, thereby increase the sensitivity of the biological analysis, and has higher stability. The bioanalytical reagent used in a heterogeneous phase is employed simply and exactly.

In order to reach the above object, the present invention apply the following technical project:

Project 1. A bioanalytical reagent, including a target detector and a signal generator, the said target detector connect the signal generator via three ways, (1) directly connected; (2) indirect coupling through a linker, spacer or adapter; (3) indirect coupling through carrier, the signal generator comprises the following formula:

(p1)y-(a trigger)_{z}-(LS)_{N}-a degradable polymer coupled with contrast agent in latent state via covalent bonds-p₂

### Wherein,

(1) Z: the number of the trigger, is a non-negative integer, Z trigger are the same or different, but only when the first trigger closed to the p1 is stimulated, the second trigger of the p1 end can be stimulated, and then the third, and so on;
(2) p1 is a protecting group for the end group of the trigger, also is the connection portion between the target detector and the signal generator, y: the number of p1, its value is 0 or 1;
(3) (LS)_{N} is self-immolative linker or spacer, the N LS are the same or different, N is a non-negative integer;
(4) p2 is a protecting group for the end group of the degradable polymer, also is the connection portion between the target detector and the signal generator;
the said signal generator is in a latent state during wash and separation procedures (contrast reagents issue a weak signal or no signal under the excitation of the external signal source), after the separation procedure, add or apply inducing matter or a condition which excites the contrast agent from a latent state to an active state into the analysis system to degrade directly the polymer or to stimulate the triggers in sequence before degrade the polymer (use the external signal when it is necessary), so, the detection signal is excited or its intensity is remarkably increased.
Project 2. A bioanalytical reagent according to project 1, wherein, the said target detector, includes the antibody or active fragment thereof, proteins, peptides, or DNA, or two or more kinds in them, or the complex formed by them, the said degradable polymer is a linear or dendritic polymeric carrier, the contrast reagents in a latent state carried on the said polymer, include fluorescent or photochromic dyes, luminescent substrate, ultrasonic reagent, MRI/PET/CT/SPECT reagent.
Project 3. A bioanalytical reagent according to project 2, where in, the signal generator is represented by the following formula, X (F)-p2, wherein,
(1) X (F) is a linear or dendritic biodegradable polymer carrying the fluorescent dye molecules through covalent bond, the fluorescent dye molecule is significantly weakened for the self-quenching or electron-withdrawing effect, the biological enzyme can degrade the polymer and eliminate the self-quenching effect, at the same time release the fluorescence;
(2) p2 is the connection portion between the target detector and the signal generator.
Project 4. A bioanalytical reagent according to project 2, where in, the signal generator is represent by the following formula, p1-X (F)-p2, wherein
(1) X (F) is a linear or dendritic biodegradable polymer carrying the fluorescent dye molecules through covalent bond, the fluorescent dye molecule is significantly weakened for the self-quenching or electron-withdrawing effect, the biological enzyme can degrade the polymer and eliminate the self-quenching effect, at the same time release the fluorescence;
(2) p1 is a protecting group for the end group of the X (F), or the connection portion between the target detector and the signal generator;
(3) p2 is a protecting group for the end group of the X (F), or the connection portion between the target detector and the signal generator.
Project 5. A usage method of the bioanalytical reagent according to project 3 or 4, wherein, during the usage process of the traditional heterophasic bioanalytical reagent, after the separation step, and before starting the external signal source (and sometimes do not need this step), add the biological enzyme which can degrade the polymer, and then start an external signal source to read the signal or read signal directly (self-generated signals, such as, the signal is light).
Project 6. A bioanalytical reagent according to project 2, wherein, the signal generator is represent by the following formula, p1-(AA)ₙ-(LS)_{N}—X (F)-p2, wherein
(1) (AA)ₙ is trigger formed by the enzyme substrate domain, n is a positive integer, enzymes cleave the signal generator between (AA)ₙ and (LS)_{N}; when (LS)_{N} does not exist, the enzymes cleave the signal generator between (AA)ₙ and X (F);
(2) (LS)_{N} is self-immolative linker or spacer, the N LS are the same or different, N is a non-negative integer;
(3) p1 is a protecting group for the end group of (AA)ₙ, also is the connection portion between the target detector and the signal generator;
(4) X (F) is a linear or dendritic biodegradable polymer carrying the fluorescent dye molecules, the fluorescent dye molecule is significantly weakened for the self-quenching or electron-withdrawing effect.
Project 7. A bioanalytical reagent according to project 6, wherein, the said linear or dendritic polymer is biological enzyme degradable polymer, the fluorescent dye connect the repeating unit of the said linear or dendritic polymer carrier by covalent bond, its fluorescence intensity is significantly weakened due to self-quenching.
Project 8. A usage method of bioanalytical reagent according to project 6, wherein, during the usage process of the traditional heterophasic bioanalytical reagent, after the separation step, and before starting the external signal source (and sometimes do not need this step), proceed the steps as follows: first, add I-type enzyme to cleave the (AA)ₙ and (LS)_{N}, or (AA)ₙ and X(F) when (LS)_{N} does not exist, then, add II-type enzyme to induce the degradation of the linear or dendritic polymer carrier followed, and then start an external signal source to read the signal or read signal directly (self-generated signals, such as, the signal is light).
Project 9. A usage method of the bioanalytical reagent according to project 8, wherein, the I-type enzyme include cysteine-containing aspartate proteolytic enzyme family (caspase family of proteases: the caspases-1, 2, 3, 6, 7, 8, 9, 10 and 12), dipeptidyl peptidase4 (DPPIV), calpain, chymotrypsin, serine protease, cathepsin (Cathepsins B, K and L), granzyme B, the SARS protease, Kallikrein, thrombin, aminopeptidase, serine aminopeptidase, tryptase, serine protease, histone deacetylase (HDACs), deacetylases (sirtuins), β-glucuronidase, β-galacosidase, lipase, esterase, protease, plasmin, carboxypeptidase G2, abzyme (also known as catalytic antibody); the II-type enzyme include biological enzyme which can cleave the lysine with unpretected ε-amine;

The following Table 1 and Table 2 list the I-type biological enzymes and the corresponding (AA)ₙ,

TABLE 1
I-type biological enzymes and the corresponding (AA)ₙ, trigger (AA)ₙ is an
amino acid or a derivative thereof,

| P1-(AA)ₙ-X | enzyme | P1-(AA)ₙ-X | enzyme |
|---|---|---|---|
| Z-DEVD-X | caspases-3 and-7 | Z-IEPD-X | granzyme B |
| Z-LETD-X | caspase-8 | Z-IETD-X | granzyme B and caspase-6 |
| GP-X | dipeptidyl peptidase 4(DPPIV) | Z-TSAVLQ-X | SARS protease |
| Z-LEHD-X | caspase-9 | Z-VNSTLQ-X | SARS protease |
| Suc-LLVY-X | calpain-and chymotrypsin-like | Z-FR-X | cathepsins B/L |
| | activities of proteasome | | |
| Z-LRR-X | trypsin-like activity of | Boc-VPR-X | kallikrein or thrombin |
| | proteasome | | |
| Z-nLPnLD-X | caspase-like activity of | Z-GGR-X | thrombin |
| | proteasome | | |
| Z-QEVY | calpain and proteasome | Z-LR-X | Cathepsin K |
| | chymotrypsin-like activity | | |
| VP-X | dipeptidyl peptidase 4(DPPIV) | Z-AAF-X | aminopeptidase |
| Z-VDVAD-X | caspase-2 | Suc-AAPF-X | serine aminopeptidase |
| Z-VEID-X | caspase-6 | Z-PRNK-X | tryptase |
| Z-ATAD-X | caspase-12 | Z-RR-X | Cathepsin B |
| Z-VAD-X | All Caspase | Z-YVAD-X | caspase-1 |
| Z-AEVD-X | caspase-10 | Z-PHE-X | Serine Protease |
| Z-LEU-X | Serine Protease | Z-LR-X | Cathepsin K |
| Z-FR-X | Cathepsin L | Ac-Lys(Ac)-X | HDACs, Sirtuins |
| Ac-K-X | Trypsin | | |

Note that, in the table 1:
Key: Z=carboxylbenzyl; Sue=succinyl; Ac=acetyl;
Boc=t-butyloxycarbamate; I=isoleucine; nL=norLeucine
All other capital letters are standard single-letter amino acid abbreviations;
p1 is protecting group of the N-terminal, these end groups can be removed in the assembly process of the signal generator, X (F) is degradable polymer carrying the contrast agent, AA is an amino acid or a derivative thereof; there are one, two or more Self-immolative Linker or Spacer between (AA)ₙ and X(F);

TABLE 2
I-type biological enzymes and the corresponding (AA)ₙ , trigger (AA)ₙ is not amino acid or derivative thereof,

| X-(AA)n | X-Linder-(AA)n | enzyme |
|---|---|---|
| 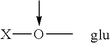 | 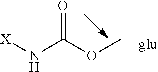 | β-glucuronidase |
| 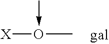 | 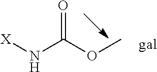 | β-galactosidase |
| 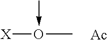 | | lipase/esterase |
| 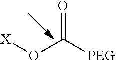 | 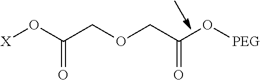 | lipase/esterase |
| 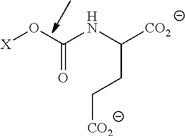 | | carboxypeptidase G2 |
| 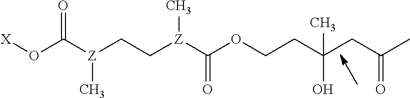 | | catalytic antibody |
| 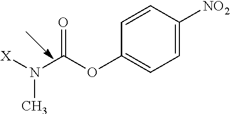 | | catalytic antibody |

Note that, in Table 2:
Key: Glu=carbohydrate glucoronide; Gal=galactose
project 10. A bioanalytical reagent according to project 7, wherein, the signal generator comprises

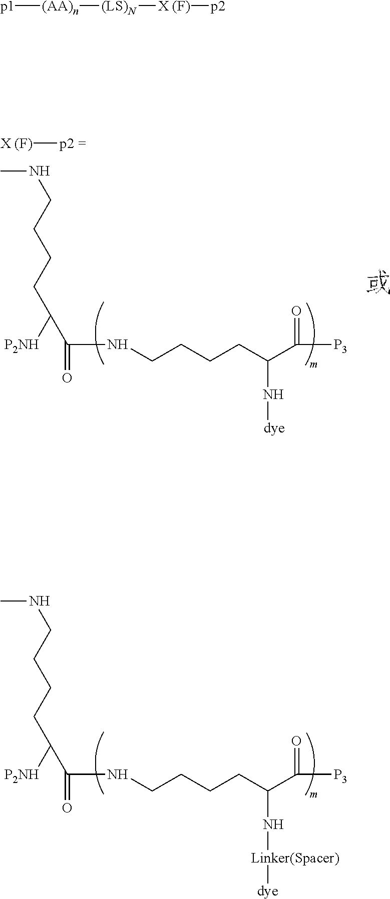

wherein:
(1) (AA)ₙ is enzyme substrate domain, n is a non-negative integer;
(2) (LS)_{N} is self-immolative linker or spacer, the N LS are the same or different, N is a non-negative integer;
(3) p1 is the end-protecting group of (AA)ₙ, can also represent the connection portion between the target detector and the signal generator;
(4) P2 is the protecting group of α-amino, including t-butyloxycarbonyl, acetyl, hexanoyl, octanoyl or benzyloxycarbonyl, or H, or dye molecules,
(5) p2=P3, is the connection portion with the target detector, or —NH₂, or other small molecules or fragment of macromolecule;
(6) the dye molecule is self-quenching dye, m is an integer greater than or equal to 1.
Project 11. A bio-analytical reagent according to project 10, wherein, trypsin cleave the lysine with unprotected ε-amino at C-terminal, result in that, the said polylysine degrade into fluorescent dye-α-lysine monomers, then, the self-quenching of dye disappears, and fluorescence releases.
Project 12. A bio-analytical reagent according to project 6, wherein, the said degradable polymer is self-immolative polymer, can automatically degrade and then, release the contrast agent in excited state.
Project 13. A bio-analytical reagent according to project 12, wherein, the fluorescent dye connect the repeating units of the linear or dendritic polymer carrier by covalent bond, its fluorescence intensity is significantly weakened due to the electron-withdrawing effect.
Project 14. A usage method of the bio-analytical reagent according to project 12, wherein, during the usage process of the traditional heterophasic bioanalytical reagent, after the separation step, and before starting the external signal source (and sometimes do not need this step), proceed the steps as follows: first, add I-type enzyme to cleave the (AA)ₙ and (LS)_{N}, or (AA)ₙ and X(F) when (LS)_{N} does not exist, then, the linear or dendritic polymer closing to it degrade and release the dye in free state, and finally, start an external signal source to read the signal or read signal directly (self-generated signals, such as, the signal is light).
Project 15. A usage method of the bioanalytical reagent according to project 14, wherein, the I-type enzyme include cysteine-containing aspartate proteolytic enzyme family (caspase family of proteases: the caspases-1, 2, 3, 6, 7, 8, 9, 10 and 12), dipeptidyl peptidase4 (DPPIV), calpain, chymotrypsin, serine protease, cathepsin (Cathepsins B, K and L), granzyme B, the SARS protease, Kallikrein, thrombin, aminopeptidase, serine aminopeptidase, tryptase, serine protease, histone deacetylase (HDACs), deacetylases (sirtuins), β-glucuronidase, β-galacosidase, lipase, esterase, protease, plasmin, carboxypeptidase G2, abzyme (also known as catalytic antibody); the following Table 1 and Table 2 list the I-type biological enzymes and the corresponding (AA)ₙ.
Project 16. A heterophasic bioanalytical reagent, wherein, the said target detector according to project 10 is antibody or active fragment thereof, the carrier is nano-particles carrying —NH₂ on the surface, the said signal generater include the following structure:

p1-(AA)ₙ-X(F)-p2,

wherein: (AA)ₙ=DEVD, p1 is Cysteine-Glycine-Glycine, the said P₂ in the X (F) is Cy7 dye, p2 is —NH₂, the dye molecule is Cy7, m=9.
Project 17. A preparation method of the heterophasic bioanalytical reagent according to project 16, wherein, the method comprises the following steps:
(1) synthesis of the signal generator according to the following procedure:

CGGDEVD-(Cy7-α-Lys)₁₀-NH₂

Polypeptide is synthetized by the 433A automatic solid-phase synthesis instrument produced by the Applied Biosystems, Inc, using solid phase polypeptide synthesis Fmoc method, the insoluble carrier resin use the Fmoc-Rink Amide TentaGel solid phase synthesis resin produced by AnaSpec, an USA Company, HBTU/HOBt (0.45 M in DMF)/DIPEA (2 M DIPEA in NMP) or HATU/DIPEA act as an activator, piperidine act as a deprotection agent; 10 times the resin (0.1 mmol), the appropriately protected amino acid (1 mmol) is contained in a small plastic bottle; The NMP is used as a solvent in the coupling process, and dichloromethane (DCM) is used to wash the solid phase resin (before and after the coupling reaction);

The process of the solid phase synthesis:

(a) Add the first amino acid to the Fmoc-Rink Amide TentaGel resin. First, remove the Fmoc group on the TentaGel resin (0.1 mmol) with 20% piperidine DMF solution, and then, wash the resin with DMF/DCM. Then, add 1 mmol Dde-Lys(Fmoc)-OH produced by BaChem company, 1 mmol DICI, and 1 mmol HOBT DMF solution into the resin solution, react at room temperature for 2-5 hours, wash the resin successively with DMF/DCM/Methanol/DCM, add the benzoic anhydride 3 mmol, and react for 30 minutes, then, wash the resin again as above mentioned.
(b) Place the following amino acids (each of the amino acids is 1 mmol) on the amino acid orbit of the ABI 433A automatic synthesizer with the following order:

(N-terminal) Fmoc-Cys(Trt)-OH, Fmoc-Gly-OH, Fmoc-Gly-OH, Fmoc-Asp(OtBu)-OH, Fmoc-Glu(OtBu)-OH, Fmoc-Val-OH, Fmoc-Asp(OtBu)-OH, [Dde-Lys(Fmoc)-OH]₁₀ (C-terminal);

After the solid phase automatic synthesis finished, treat the resin with 2% Hydrazine solution to deprotect the Dde group in the Dde-Lys(Fmoc)-OH. Add the 30 mmol Cy7-NHS produced by the GE Healthcare company into the above resin, after the reaction for 1 hour, wash the resin successively with DMF/DCM;

The polymer is cutted off the resin under argon protection: per 100 mg resin carrying polypeptide, add 1 ml of a mixture of the following proportions: (TFA:water:Tis=95:2.5:2.5); The solution of the resin mixture is then shaking for 2 hours at room temperature. Subsequently, the mixture solution is filtered to remove the resin, and then added dropwise into the ice-cold diethyl ether to precipitate out the polypeptide, through repeated centrifugation and washing, and finally, the polypeptide is dried;

the full name of the abbreviation used in the above step is as follows:

### TIS: Triisopropylsilane

TFA: Trifluoroacetic acid

### HOBt: 1-Hydroxylformamide

### DMF: N,N-Dimethylformamide

### DCM: Dicholoromethane

### DIPEA (DIEA): N,N-Diisopropylethylamine

### NMP: N-methylpyrrolidone

HATU: 2-(7-Aza-1H-benzotriazole-1-yl)-1,1,3,3-tetramethyluroniumHexafluorophosphate
HBTU: 2-(1H-Benzotriazole-1-yl)-1,1,3,3-tetramethyluroniumHexafluorophosphate

### DICI: N,N′-Diisopropylcarbodiimide

(2) the said —NH₂ on the nano particles is transformed into maleimide by Succinimidyl-4-(N-maleimidomethyl) cyclohexane-1-carboxylate (SMCC), or
Sulfosuccinimidyl-4-(N-maleimidomethyl) cyclohexane-1-carboxylate (Sulfo-SMCC), and then removing the excess SMCC or Sulfo-SMCC;
(3) restore the disulfide bond at its own hinge of the complete antibody molecules by excess dithiothreitol (DTT) to produce —SH functional group, and then removing the excess DTT;
(4) The product of step (3) is added to the product of step (2), coupling reaction between the —SH functional group produced in step (3) and the maleimide on the nano particles produced in step (2) occur in the buffer with pH 6-8, the molar ratio of the product of step (3) and the product of step (2) is: X:1 (X=2-20), stirred for 10-120 minutes; the product (previously dissolved in deionized water) of step (1) is then added, the molar ratio of the product of step (1) and the product of step (2) is: Y:1 (Y=2-100), and stirred for 10-300 minutes, and finally add an excess hydrotropic compound containing maleimide functional group, such as PEG-Maleimide, Sulfo-Maleimide, slight stirred for 10-300 minutes at 2-8° C.;
(5) remove the free antibody in the product of step (4), free product of step (1), the excess hydrotropic compound containing maleimide functional group, and the other residues of small molecule compounds, to obtain the target product: Antibodies—carrier—latent state fluorescence signal generator system, and then add stabilizing agent into the target product, store the product at low temperature.
Project 18. A heterophasic bioanalytical reagent, wherein, the said target detector in project 10 is antibody or active fragment thereof, the carrier is nano-particles with —NH₂ on its surface, the said signal generator include the following structure:

p1-(AA)ₙ-(LS)_{N}—X(F)-p2

wherein, (AA)ₙ=DEVD, p1 is Cysteine-Glycine-Glycine, the said P2 in X (F) structure is Cy7 dye, p2 is —NH₂, the dye is Cy7, m=9,

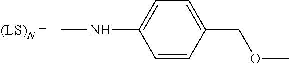

Project 19. A preparation method of the heterophase bioanalytical reagent in project 18, wherein, the method comprises the following steps:

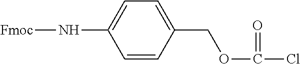

### (1) Preparation

add 10 equivalents of DIPEA with phosgene (20% phosgene in toluene solution, Sigma) into the 1 equivalent of Fmoc-4-aminobenzylalcohol (Anaspec of USA) DMF, react overnight under nitrogen protection, then remove the solvent by vacuum, and then proceed the separation and purification by flash chromatographic;
(2) the said signal generator is synthesized according to the following procedure:

CGGDEVD-(LS)_{N}-(Cy7-α-Lys)₁₀-NH₂

The process of the solid phase synthesis:

(a) Add the first amino acid to the Fmoc-Rink Amide TentaGel resin according to project 12;
(b) place the following amino acids (each of the amino acids is 1 mmol) on the amino acid orbit of the ABI 433A automatic synthesizer with the following order:

(N-terminal) [Dde-Lys(Fmoc)-OH]₁₀ (C-terminal); proceed the automatic solid phase synthesis by machine; then treat the resin with 20% piperdine solution to deprotect, add 10 equivalents of compound prepared in step (1) compared to the active point of the resin, and 10 equivalents of DIPEA, react 2-5 hours at room temperature, then wash the resin, and continue to add the following amino acid by machine (start form deprotection):

(N-terminal) Fmoc-Cys(Trt)-OH, Fmoc-Gly-OH, Fmoc-Gly-OH, Fmoc-Asp(OtBu)-OH, Fmoc-Glu(OtBu)-OH, Fmoc-Val-OH, Fmoc-Asp(OtBu)-OH (C-terminal);

After the solid phase automatic synthesis finished, treat the resin with 2% Hydrazine solution to deprotect the Dde group in the Dde-Lys(Fmoc)-OH; add the 30 mmol Cy7-NHS produced by the GE Healthcare company into the above resin, after the reaction for 1 hour, wash the resin successively with DMF/DCM; the sequential treat is the same as that in project 18;

(3) the said —NH₂ on the nano particles is transformed into maleimide by SMCC or Sulfo-SMCC, and then removing the excess SMCC or Sulfo-SMCC;
(4) restore the disulfide bond at its own hinge of the complete antibody molecules by excess dithiothreitol (DTT) to produce —SH functional group, and then removing the excess DTT;
(5) The product of step (4) is added to the product of step (3), coupling reaction between the —SH functional group produced in step (4) and the maleimide on the nano particles produced in step (3) occur in the buffer with pH 6-8, the molar ratio of the product of step (4) and the product of step (3) is: X:1 (X=2-20), stirred for 10-120 minutes; the product (previously dissolved in deionized water) of step (2) is then added, the molar ratio of the product of step (2) and the product of step (3) is: Y:1 (Y=2-100), and stirred for 10-300 minutes, and finally add excess hydrotropic compound containing maleimide functional group, such as PEG-Maleimide, Sulfo-Maleimide, slight stirred for 10-300 minutes at 2-8° C.;
(6) remove the free antibody in the product of step (4), free product of step (1), the excess hydrotropic compound containing maleimide functional group, and the other residues of small molecule compounds, to obtain the target product: Antibodies—carrier—p1-(AA)ₙ-(LS)_{N}—X(F)-p2 system, and then add stabilizing agent into the target product, store the product at low temperature.
Project 20. An kit (package) of the signal generator according to project 1, including the usage of biological enzyme and the corresponding (AA)ₙ listed in the table 1 and table 2.

Compared with the prior art, contrast agent (also known as the signal generating unit) in the heterophasic bioanalytical reagent provided by the present invention can not send a strong signal directly in the action of the external signal generating source, it is necessary to degrade the polymer carrying many contrast agent by further applied induced matter or changing the testing conditions, the contrast agent can send a strong signal after it is in the free state, we call that as the signal generation unit enter into the excited state from the latent state. Therefore, the individual molecules of contrast agent in the heterophasic bioanalytical reagent provided by the present invention do not influence each other, and be able to send a stronger signal, thus improving the sensitivity of the biological detection analysis, and has high stability. The heterophasic bioanalytical reagent is employed simply and exactly.

When the said contrast agent is a fluorescent dye, the heterophasic bioanalytical reagent of the present invention has two advantages, one is not limited to the self-quenching of the fluorescent dye, can carry as much dye molecules as possible, the other advantage is able to suppress the Auto-Bleaching of the dye molecules.

### BRIEF DESCRIPTION OF THE DRAWINGS

FIG. 1 shows the theory schematic diagram of the existing heterophasic biological analysis reagent;

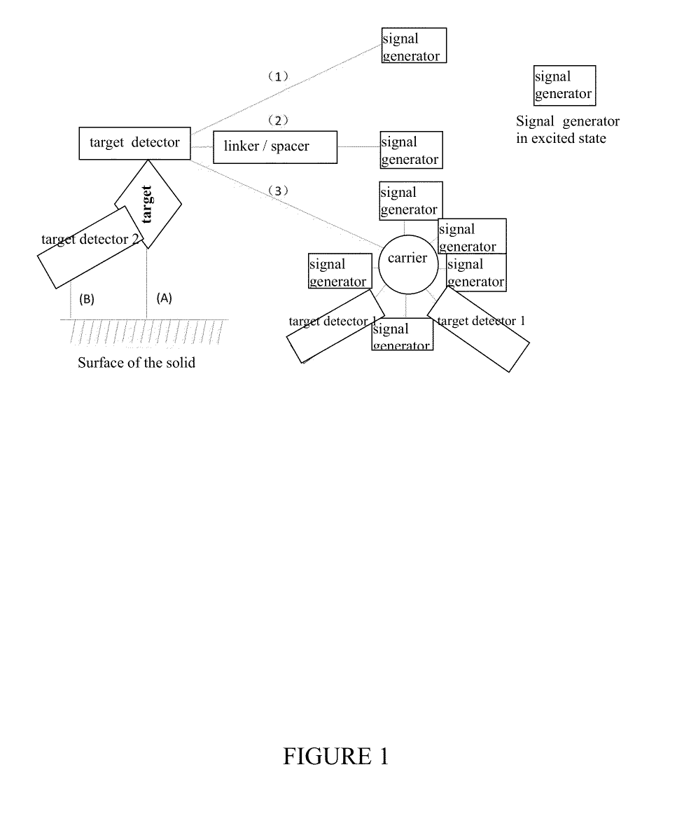

FIG. 2 shows the structure of the signal generator of the present invention, and the difference is the usage method with that of the existing signal generator;

FIG. 3 shows the action mechanism of the signal generator when the polymer in the signal generator of the present invention is enzyme degradable;

FIG. 4 shows the action mechanism of the signal generator when the polymer in the signal generator of the present invention is self-degradable;

FIG. 5 shows the testing result of the heterophasic bioanalytical reagent of the present invention.

### DESCRIPTION OF THE PREFERRED EMBODIMENTS

The contrast agent (also known as the signal generating unit) in the heterophasic bioanalytical reagent provided by the present invention can not send a strong signal directly under the action of the external signal generating source, it is necessary to degrade the polymer carrying many contrast agent by further applied induced matter or changing the testing conditions, the contrast agent can send a strong signal after it is in the free state, we call that as the signal generation unit enter into the excited state from the latent state.

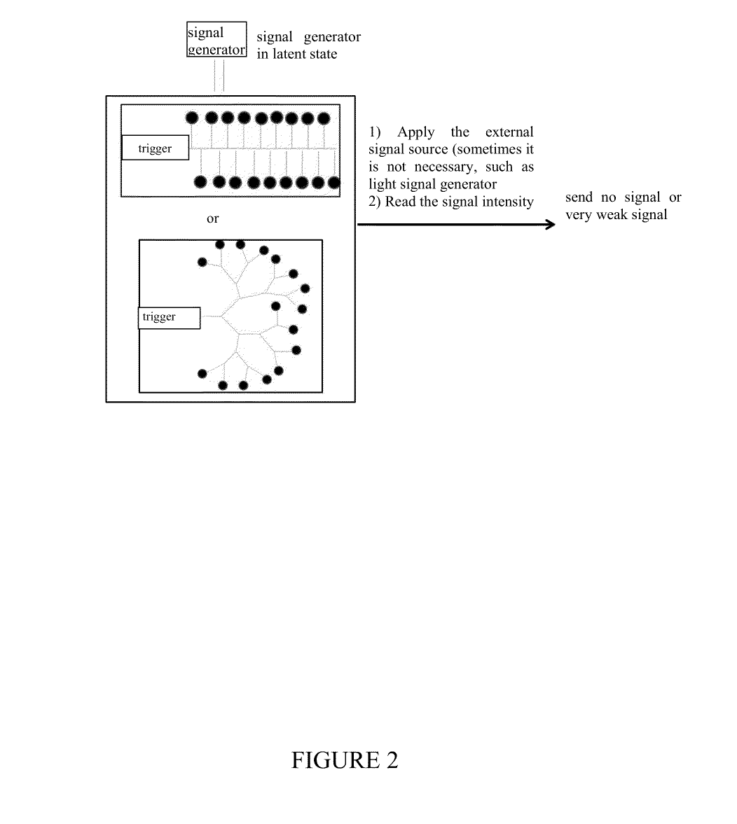

As shown in FIG. 2, the signal generator is in a latent state, the contrast agent do not send signal or send very weak signal;

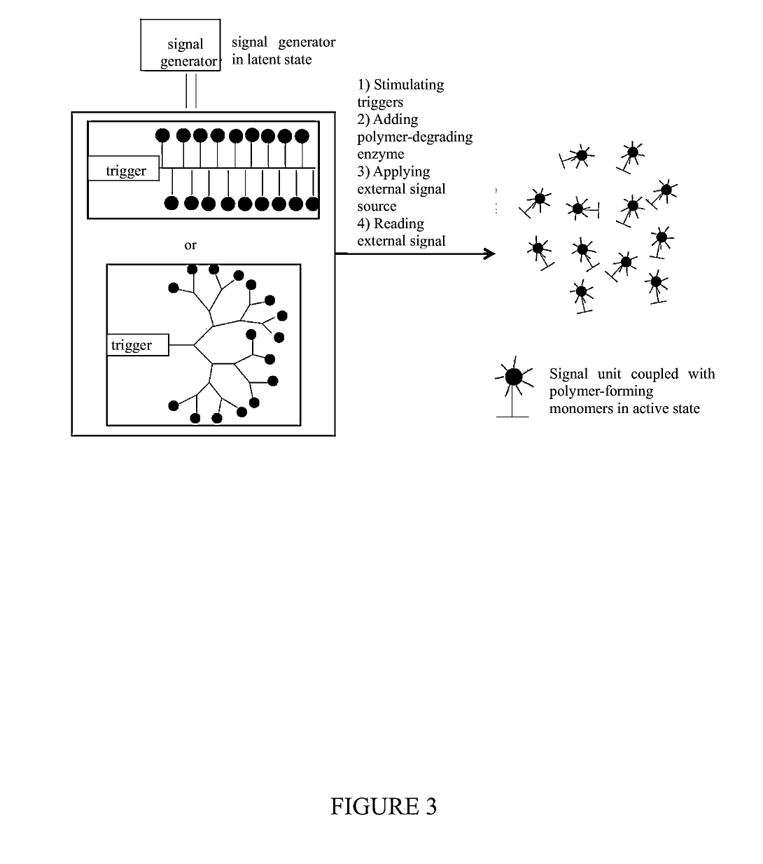

As shown in FIG. 3, when the polymer in the signal generator of the invention is enzyme degradable polymer, the usage method of the heterophasic bioanalytical reagent is as follows: during the usage process of the heterogeneous bioanalytical reagent, after the separation step, add the II-type enzyme directly, the II-type enzyme includes trypsin, the trypsin cleave the lysine with unprotected ε-amino at C-terminal, result in that, the said polylysine is degraded into fluorescent dye-α-lysine monomers, then, the self-quenching of dye disappears, and fluorescence releases. and then start an external signal source to read the signal or read signal directly (self-generated signals, such as, the signal is light).

When (AA)ₙ exist, during the usage process of the heterophasic bioanalytical reagent, after the separation step, first, add I-type enzyme to cleave the (AA)ₙ and (LS)_{N}, or (AA)ₙ and X(F), then, add II-type enzyme, then the polylysine is degraded into fluorescent dye-α-lysine monomers, therefore the self-quenching of dye disappears, and fluorescence releases. and then start an external signal source to read the signal or read signal directly (self-generated signals, such as, the signal is light).

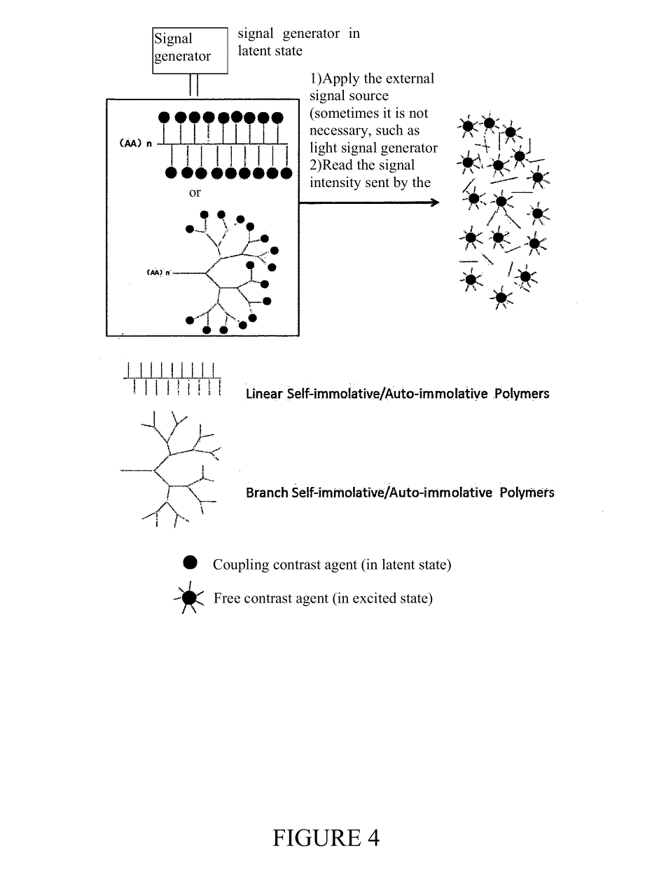

As shown in FIG. 4, when the polymer in the signal generator of the invention is self-immolative polymer, first, add I-type enzyme to cleave the (AA)ₙ and (LS)_{N}, or (AA)ₙ and X(F), then, the linear or dendritic self-immolative polymer closing to it automatically degrade and release the dye in free state, and then, start an external signal source to read the signal or read signal directly (self-generated signals, such as, the signal is light).

### Example 1

The synthesis of the

(Goat-anti-rabbit IgG)x-nanoparticle-[CGGDEVD-(Cy7-α-Lys)₁₀-NH₂]_{y}

Wherein, X is 2-6, y is 4-10. The synthesis of the heterophasic bioanalytical reagent, comprising the steps as below:
(1) Synthesize signal generator according to project 12:

CGGDEVD-(Cy7-α-Lys)₁₀-NH₂.

(2) Add 400 times SMCC dissolved in DMSO into 1×PBS solution with nano-particles, the nano-particles carry —NH₂ on its surface, and the diameter of the nano-particle is 16 nm, after the reaction for 2 hours, using PD30 column (Amersham Biosciences) to remove the excess SMCC;
(3) The complete goat-anti-rabbit IgG is formulated into 5 mg/mL solution by 1M dithiothreitol (DTT) aqueous solution, shaking at room temperature for 10 minutes, the excess DTT is removed using PD-10 column (Amersham Biosciences);
(4) The product of step (3) is added to the product of step (2), the molar ratio of the product of step (3) and the product of step (2) is 10:1, adjust the pH of the reaction system to 6-8, and the mixture is stirred for 120 minutes; the product (predissolved in deionized water) of step (1) is then added, the molar ratio of the product of step (1) and the product of step (2) is: 20:1, stirred for 2 hours, then add excess Sulfo-Maleimide, slight stirred for 300 minutes at 2-8° C.;
(5) The product of the steps (4) flow through a Superdex 200, to collect the target product:

(Goat-anti-rabbit IgG)ₓ-Nanoparticles-[CGGDEVD-(Cy7-α-Lys)₁₀-NH₂]_{y}

then add additives which can stabilize the target into the target product, store this product at low temperature.

### Example 2

The (Goat-anti-rabbit IgG)ₓ-nanoparticles-[CGGDEVD-(Cy7-α-Lys)₁₀-NH₂]_{y} in the example 1 is used for the analysis and detection of antigen, the usage method comprises the following steps:

(1) the rabbit IgG is diluted to 100 ng/μL, 10 ng/μL, 1 ng/μL, 0.1 ng/μL, then add dropwise 1 μL solution of 100 ng/μL, 10 ng/μL, 1 ng/μL, 0.1 ng/μL using a dropper onto the nitrocellulose membrane, dried at room temperature to obtain the four IgG dot with a diameter of approximately 2-4 mm, and four dots, respectively, containing 100 ng, 10 ng, 1 ng and 0.1 ng IgG.
(2) the Rabbit immunoglobulin nitrocellulose membrane of the step (1) is placed in the 10 ml of 5% (percentage by weight) milk in PBS buffer, shaking for 10 minutes, then wash the above immunoglobulin film with the above PBS, repeat 3 times, and then remove the buffer, the nitrocellulose membrane is reserved.
(3) the Rabbit immunoglobulin nitrocellulose membrane of the Step (2) is placed in the heterophasic bioanalytical reagent containing 15 pmol (nanoparticles) of example 1 and 10 ml of 5% milk in PBS buffer, reacted for 30 minutes, and then remove the buffer, the nitrocellulose membrane is reserved.
(4) the film of step (3) is soaked in the 5% (percentage by weight) milk in 1×PBS buffer for 10 minutes, then remove the PBS buffer; repeat the washing step 3 times.
(5) prepare caspase 3 solution:

Axxora human recombinant and Lyophilized active caspase-3 is dissolved in the 15% glycerol in PBS solution, and then diluted in 50 mM HEPES, pH 7.2, 50 mM NaCl, 0.1% CHAPS, 10 mM EDTA, 5% glycerol and 10 mM DTT solution, to obtain 1 U/μl of caspase 3 solution.

(6) the solution of step (5) is added dropwise to the position of the immunoglobulin nitrocellulose IgG original point of step (4), 5 μl caspase 3 solution is added onto the each IgG original point (divided into 5 times, each time add 1 μl, 5 minute intervals between two times), then 5 μl 1×PBS solution containing excess Trypsin is added onto the each IgG original point by the same method. Finally, the immunoglobulin nitrocellulose membrane is placed within the Kodak vivo imaging instrument, to obtain the fluorescence imaging by the suitable optical filters.
(7) proceed the ROI analysis of the image.

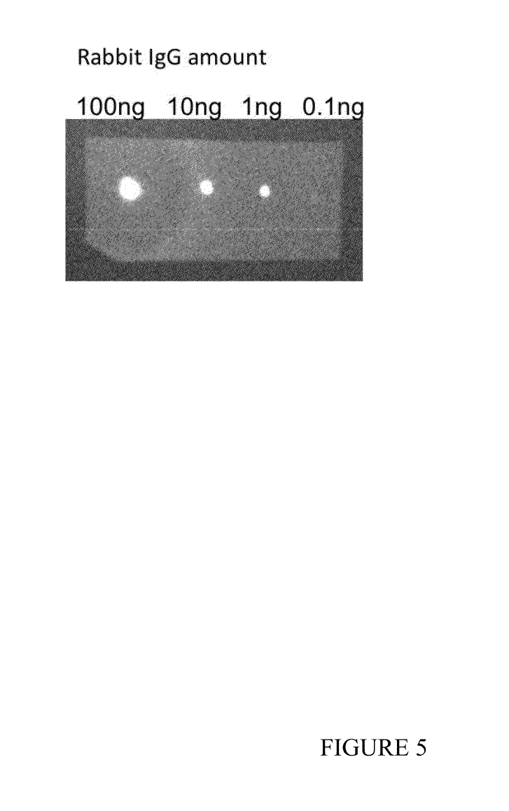

The test results are shown in FIG. 5.

The above described is the preferred embodiment of the present invention only, and not intended to limit the scope of protection of the present invention. All modifications and alterations made to the content of the present invention, are covered within the protection scope of the present invention.

We claim:

1. A bioanalytical reagent, including a target detector and a signal generator, the said target detector connect the signal generator via three ways, (1) directly connected; (2) indirect coupling through a linker, spacer or adapter; (3) indirect coupling through carrier, wherein, the signal generator comprises the following formula:
(p1)y-(a trigger)_{z}-(LS)_{N}-a degradable polymer coupled with contrast agent in latent state via covalent bonds-p₂
Wherein,
(1) Z: the number of the trigger, is a non-negative integer, Z trigger are the same or different, but only when the first trigger closed to the p1 is stimulated, the second trigger of the p1 end can be stimulated, and then the third, and so on;
(2) p1 is a protecting group for the end group of the trigger, also is the connection portion between the target detector and the signal generator, y: the number of p1, its value is 0 or 1;
(3) (LS)_{N} is self-immolative linker or spacer, the N LS are the same or different, N is a non-negative integer;
(4) p2 is a protecting group for the end group of the degradable polymer, also is the connection portion between the target detector and the signal generator;
the said signal generator is in a latent state during wash and separation procedures (contrast reagents issue a weak signal or no signal under the excitation of the external signal source), after the separation procedure, add or apply inducing matter or a condition which excites the contrast agent from a latent state to an active state into the analysis system to degrade directly the polymer or to stimulate the triggers in sequence before degrade the polymer (use the external signal when it is necessary), so, the detection signal is excited or its intensity is remarkably increased.

2. A bioanalytical reagent according to claim 1, wherein, the said target detector, includes the antibody or active fragment thereof, proteins, peptides, or DNA, or two or more kinds in them, or the complex formed by them, the said degradable polymer is a linear or dendritic polymeric carrier, the contrast reagents in a latent state carried on the said polymer, include fluorescent or photochromic dyes, luminescent substrate, ultrasonic reagent, MRI/PET/CT/SPECT reagent.

3. A bioanalytical reagent according to claim 2, where in, the signal generator is represented by the following formula, X (F)-p2, wherein,
(1) X (F) is a linear or dendritic biodegradable polymer carrying the fluorescent dye molecules through covalent bond, the fluorescent dye molecule is significantly weakened for the self-quenching or electron-withdrawing effect, the biological enzyme can degrade the polymer and eliminate the self-quenching effect, at the same time release the fluorescence;
(2) p2 is the connection portion between the target detector and the signal generator.

4. A bioanalytical reagent according to claim 2, where in, the signal generator is represent by the following formula, p1-X (F)-p2, wherein
(1) X (F) is a linear or dendritic biodegradable polymer carrying the fluorescent dye molecules through covalent bond, the fluorescent dye molecule is significantly weakened for the self-quenching or electron-withdrawing effect, the biological enzyme can degrade the polymer and eliminate the self-quenching effect, at the same time release the fluorescence;
(2) p1 is a protecting group for the end group of the X (F), or the connection portion between the target detector and the signal generator;
(3) p2 is a protecting group for the end group of the X (F), or the connection portion between the target detector and the signal generator.

5. A usage method of the bioanalytical reagent according to claim 3 or 4, wherein, during the usage process of the traditional heterophasic bioanalytical reagent, after the separation step, and before starting the external signal source (and sometimes do not need this step), add the biological enzyme which can degrade the polymer, and then start an external signal source to read the signal or read signal directly (self-generated signals, such as, the signal is light).

6. A bioanalytical reagent according to claim 2, wherein, the signal generator is represent by the following formula, p1-(AA)ₙ-(LS)_{N}—X(F)-p2, wherein
(1) (AA)ₙ is trigger formed by the enzyme substrate domain, n is a positive integer, enzymes cleave the signal generator between (AA)ₙ and (LS)_{N}; when (LS)_{N} does not exist, the enzymes cleave the signal generator between (AA)ₙ and X (F);
(2) (LS)_{N} is self-immolative linker or spacer, the N LS are the same or different, N is a non-negative integer;
(3) p1 is a protecting group for the end group of (AA)ₙ, also is the connection portion between the target detector and the signal generator;
(4) X (F) is a linear or dendritic biodegradable polymer carrying the fluorescent dye molecules, the fluorescent dye molecule is significantly weakened for the self-quenching or electron-withdrawing effect.

7. A bioanalytical reagent according to claim 6, wherein, the said linear or dendritic polymer is biological enzyme degradable polymer, the fluorescent dye connect the repeating unit of the said linear or dendritic polymer carrier by covalent bond, its fluorescence intensity is significantly weakened due to self-quenching.

8. A usage method of bioanalytical reagent according to claim 6, wherein, during the usage process of the traditional heterophasic bioanalytical reagent, after the separation step, and before starting the external signal source (and sometimes do not need this step), proceed the steps as follows: first, add I-type enzyme to cleave the (AA)ₙ and (LS)_{N}, or (AA)ₙ and X(F) when (LS)_{N} does not exist, then, add II-type enzyme to induce the degradation of the linear or dendritic polymer carrier followed, and then start an external signal source to read the signal or read signal directly (self-generated signals, such as, the signal is light).

9. A usage method of the bioanalytical reagent according to claim 8, wherein, the I-type enzyme include cysteine-containing aspartate proteolytic enzyme family (caspase family of proteases: the caspases-1, 2, 3, 6, 7, 8, 9, 10 and 12), dipeptidyl peptidase4 (DPPIV), calpain, chymotrypsin, serine protease, cathepsin (Cathepsins B, K and L), granzyme B, the SARS protease, Kallikrein, thrombin, aminopeptidase, serine aminopeptidase, tryptase, serine protease, histone deacetylase (HDACs), deacetylases (sirtuins), β-glucuronidase, β-galacosidase, lipase, esterase, protease, plasmin, carboxypeptidase G2, abzyme (also known as catalytic antibody); the II-type enzyme include biological enzyme which can cleave the lysine with unpretected ε-amine;
The following Table 1 and Table 2 list the I-type biological enzymes and the corresponding (AA)ₙ,
TABLE 1
I-type biological enzymes and the corresponding (AA)ₙ, trigger (AA)ₙ is an
amino acid or a derivative thereof,

| P1-(AA)ₙ-X | enzyme | P1-(AA)ₙ-X | enzyme |
|---|---|---|---|
| Z-DEVD-X | caspases-3 and-7 | Z-IEPD-X | granzyme B |
| Z-LETD-X | caspase-8 | Z-IETD-X | granzyme B and caspase-6 |
| GP-X | dipeptidyl peptidase 4(DPPIV) | Z-TSAVLQ-X | SARS protease |
| Z-LEHD-X | caspase-9 | Z-VNSTLQ-X | SARS protease |
| Suc-LLVY-X | calpain-and chymotrypsin-like | Z-FR-X | cathepsins B/L |
| | activities of proteasome | | |
| Z-LRR-X | trypsin-like activity of | Boc-VPR-X | kallikrein or thrombin |
| | proteasome | | |
| Z-nLPnLD-X | caspase-like activity of | Z-GGR-X | thrombin |
| | proteasome | | |
| Z-QEVY | calpain and proteasome | Z-LR-X | Cathepsin K |
| | chymotrypsin-like activity | | |
| VP-X | dipeptidyl peptidase 4(DPPIV) | Z-AAF-X | aminopeptidase |
| Z-VDVAD-X | caspase-2 | Suc-AAPF-X | serine aminopeptidase |
| Z-VEID-X | caspase-6 | Z-PRNK-X | tryptase |
| Z-ATAD-X | caspase-12 | Z-RR-X | Cathepsin B |
| Z-VAD-X | All Caspase | Z-YVAD-X | caspase-1 |
| Z-AEVD-X | caspase-10 | Z-PHE-X | Serine Protease |
| Z-LEU-X | Serine Protease | Z-LR-X | Cathepsin K |
| Z-FR-X | Cathepsin L | Ac-Lys(Ac)-X | HDACs, Sirtuins |
| Ac-K-X | Trypsin | | |

Note that, in the table 1:
Key: Z=carboxylbenzyl; Sue=succinyl; Ac=acetyl;
Boc=t-butyloxycarbamate; I=isoleucine; nL=norLeucine
All other capital letters are standard single-letter amino acid abbreviations;
p1 is protecting group of the N-terminal, these end groups can be removed in the assembly process of the signal generator, X (F) is degradable polymer carrying the contrast agent, AA is an amino acid or a derivative thereof; there are one, two or more Self-immolative Linker or Spacer between (AA)ₙ and X(F);
TABLE 2
I-type biological enzymes and the corresponding (AA)ₙ , trigger (AA)ₙ is not amino acid or derivative thereof,

| X-(AA)n | X-Linder-(AA)n | enzyme |
|---|---|---|
| 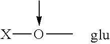 | 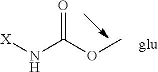 | β-glucuronidase |
| 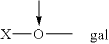 | 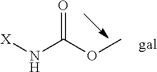 | β-galactosidase |
| 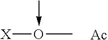 | | lipase/esterase |
| 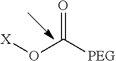 | 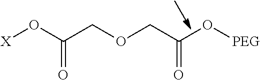 | lipase/esterase |
| 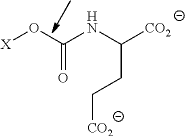 | | carboxypeptidase G2 |
| 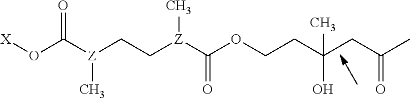 | | catalytic antibody |
| 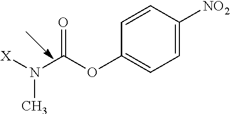 | | catalytic antibody |

Note that, in Table 2:
Key: Glu=carbohydrate glucoronide; Gal=galactose

10. A bioanalytical reagent according to claim 7, wherein, the signal generator comprises 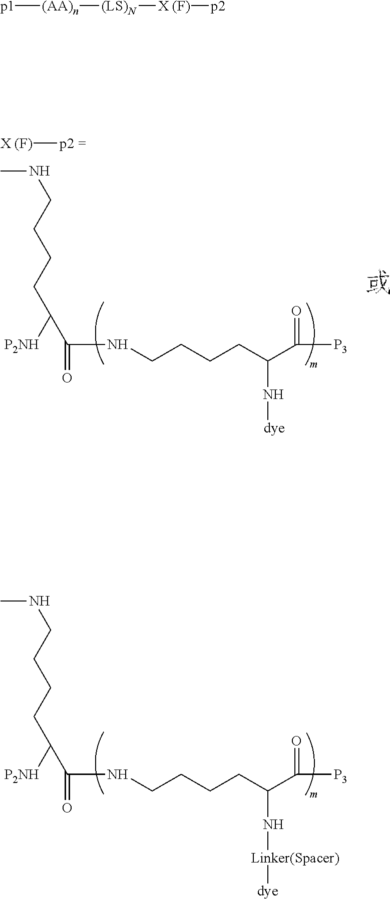
wherein:
(1) (AA)ₙ is enzyme substrate domain, n is a non-negative integer;
(2) (LS)_{N} is self-immolative linker or spacer, the N LS are the same or different, N is a non-negative integer;
(3) p1 is the end-protecting group of (AA)ₙ, can also represent the connection portion between the target detector and the signal generator;
(4) P2 is the protecting group of α-amino, including t-butyloxycarbonyl, acetyl, hexanoyl, octanoyl or benzyloxycarbonyl, or H, or dye molecules,
(5) p2=P3, is the connection portion with the target detector, or —NH₂, or other small molecules or fragment of macromolecule;
(6) the dye molecule is self-quenching dye, m is an integer greater than or equal to 1.

11. A bio-analytical reagent according to claim 10, wherein, trypsin cleave the lysine with unprotected ε-amino at C-terminal, result in that, the said polylysine degrade into fluorescent dye-α-lysine monomers, then, the self-quenching of dye disappears, and fluorescence releases.

12. A bio-analytical reagent according to claim 6, wherein, the said degradable polymer is self-immolative polymer, can automatically degrade and then, release the contrast agent in excited state.

13. A bio-analytical reagent according to claim 12, wherein, the fluorescent dye connect the repeating units of the linear or dendritic polymer carrier by covalent bond, its fluorescence intensity is significantly weakened due to the electron-withdrawing effect.

14. A usage method of the bio-analytical reagent according to claim 12, wherein, during the usage process of the traditional heterophasic bioanalytical reagent, after the separation step, and before starting the external signal source (and sometimes do not need this step), proceed the steps as follows: first, add I-type enzyme to cleave the (AA)ₙ and (LS)_{N}, or (AA)ₙ and X(F) when (LS)_{N} does not exist, then, the linear or dendritic polymer closing to it degrade and release the dye in free state, and finally, start an external signal source to read the signal or read signal directly (self-generated signals, such as, the signal is light).

15. A usage method of the bioanalytical reagent according to claim 14, wherein, the I-type enzyme include cysteine-containing aspartate proteolytic enzyme family (caspase family of proteases: the caspases-1, 2, 3, 6, 7, 8, 9, 10 and 12), dipeptidyl peptidase4 (DPPIV), calpain, chymotrypsin, serine protease, cathepsin (Cathepsins B, K and L), granzyme B, the SARS protease, Kallikrein, thrombin, aminopeptidase, serine aminopeptidase, tryptase, serine protease, histone deacetylase (HDACs), deacetylases (sirtuins), β-glucuronidase, β-galacosidase, lipase, esterase, protease, plasmin, carboxypeptidase G2, abzyme (also known as catalytic antibody); the following Table 1 and Table 2 list the I-type biological enzymes and the corresponding (AA)ₙ.

16. A heterophasic bioanalytical reagent, wherein, the said target detector according to claim 10 is antibody or active fragment thereof, the carrier is nano-particles carrying —NH₂ on the surface, the said signal generater include the following structure:
p1-(AA)ₙ-X(F)-p2,
wherein: (AA)ₙ=DEVD, p1 is Cysteine-Glycine-Glycine, the said P₂ in the X (F) is Cy7 dye, p2 is —NH₂, the dye molecule is Cy7, m=9.

17. A preparation method of the heterophasic bioanalytical reagent according to claim 16, wherein, the method comprises the following steps:
(1) synthesis of the signal generator according to the following procedure:
CGGDEVD-(Cy7-α-Lys)₁₀-NH₂
Polypeptide is synthetized by the 433A automatic solid-phase synthesis instrument produced by the Applied Biosystems, Inc, using solid phase polypeptide synthesis Fmoc method, the insoluble carrier resin use the Fmoc-Rink Amide TentaGel solid phase synthesis resin produced by AnaSpec, an USA Company, HBTU/HOBt (0.45 M in DMF)/DIPEA (2 M DIPEA in NMP) or HATU/DIPEA act as an activator, piperidine act as a deprotection agent; 10 times the resin (0.1 mmol), the appropriately protected amino acid (1 mmol) is contained in a small plastic bottle. The NMP is used as a solvent in the coupling process, and dichloromethane (DCM) is used to wash the solid phase resin (before and after the coupling reaction).
The process of the solid phase synthesis:
(a) Add the first amino acid to the Fmoc-Rink Amide TentaGel resin. First, remove the Fmoc group on the TentaGel resin (0.1 mmol) with 20% piperidine DMF solution, and then, wash the resin with DMF/DCM. Then, add 1 mmol Dde-Lys(Fmoc)-OH produced by BaChem company, 1 mmol DICI, and 1 mmol HOBT DMF solution into the resin solution, react at room temperature for 2-5 hours, wash the resin successively with DMF/DCM/Methanol/DCM, add the benzoic anhydride 3 mmol, and react for 30 minutes, then, wash the resin again as above mentioned.
(b) Place the following amino acids (each of the amino acids is 1 mmol) on the amino acid orbit of the ABI 433A automatic synthesizer with the following order:
(N-terminal) Fmoc-Cys(Trt)-OH, Fmoc-Gly-OH, Fmoc-Gly-OH, Fmoc-Asp(OtBu)-OH, Fmoc-Glu(OtBu)-OH, Fmoc-Val-OH, Fmoc-Asp(OtBu)-OH, [Dde-Lys(Fmoc)-OH]₁₀ (C-terminal);
After the solid phase automatic synthesis finished, treat the resin with 2% Hydrazine solution to deprotect the Dde group in the Dde-Lys(Fmoc)-OH. Add the 30 mmol Cy7-NHS produced by the GE Healthcare company into the above resin, after the reaction for 1 hour, wash the resin successively with DMF/DCM;
The polymer is culled off the resin under argon protection: per 100 mg resin carrying polypeptide, add 1 ml of a mixture of the following proportions: (TFA:water:Tis=95:2.5:2.5); The solution of the resin mixture is then shaking for 2 hours at room temperature. Subsequently, the mixture solution is filtered to remove the resin, and then added dropwise into the ice-cold diethyl ether to precipitate out the polypeptide, through repeated centrifugation and washing, and finally, the polypeptide is dried;
the full name of the abbreviation used in the above step is as follows:
TIS: Triisopropylsilane
TFA: Trifluoroacetic acid
HOBt: 1-Hydroxylformamide
DMF: N,N-Dimethylformamide
DCM: Dicholoromethane
DIPEA (DIEA): N,N-Diisopropylethylamine
NMP: N-methylpyrrolidone
HATU:
2-(7-Aza-1H-benzotriazole-1-yl)-1,1,3,3-tetramethyluroniumHexafluorophosphate
HBTU: 2-(1H-Benzotriazole-1-yl)-1,1,3,3-tetramethyluroniumHexafluorophosphate
DICE N,N′-Diisopropylcarbodiimide
(2) the said —NH₂ on the nano particles is transformed into maleimide by Succinimidyl-4-(N-maleimidomethyl) cyclohexane-1-carboxylate (SMCC), or
Sulfosuccinimidyl-4-(N-maleimidomethyl) cyclohexane-1-carboxylate (Sulfo-SMCC), and then removing the excess SMCC or Sulfo-SMCC;
(3) restore the disulfide bond at its own hinge of the complete antibody molecules by excess dithiothreitol (DTT) to produce —SH functional group, and then removing the excess DTT;
(4) The product of step (3) is added to the product of step (2), coupling reaction between the —SH functional group produced in step (3) and the maleimide on the nano particles produced in step (2) occur in the buffer with pH 6-8, the molar ratio of the product of step (3) and the product of step (2) is: X:1 (X=2-20), stirred for 10-120 minutes; the product (previously dissolved in deionized water) of step (1) is then added, the molar ratio of the product of step (1) and the product of step (2) is: Y:1 (Y=2-100), and stirred for 10-300 minutes, and finally add an excess hydrotropic compound containing maleimide functional group, such as PEG-Maleimide, Sulfo-Maleimide, slight stirred for 10-300 minutes at 2-8° C.;
(5) remove the free antibody in the product of step (4), free product of step (1), the excess hydrotropic compound containing maleimide functional group, and the other residues of small molecule compounds, to obtain the target product: Antibodies—carrier—latent state fluorescence signal generator system, and then add stabilizing agent into the target product, store the product at low temperature.

18. A heterophasic bioanalytical reagent, wherein, the said target detector in claim 10 is antibody or active fragment thereof, the carrier is nano-particles with —NH₂ on its surface, the said signal generator include the following structure:
p1-(AA)ₙ-(LS)_{N}—X(F)-p2
wherein, (AA)ₙ=DEVD, p1 is Cysteine-Glycine-Glycine, the said P2 in X (F) structure is Cy7 dye, p2 is —NH₂, the dye is Cy7, m=9,

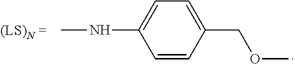

19. A preparation method of the heterophase bioanalytical reagent in claim 18, wherein, the method comprises the following steps: 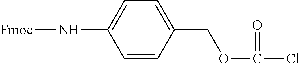
(1) Preparation
add 10 equivalents of DIPEA with phosgene (20% phosgene in toluene solution, Sigma) into the 1 equivalent of Fmoc-4-aminobenzylalcohol (Anaspec of USA) DMF, react overnight under nitrogen protection, then remove the solvent by vacuum, and then proceed the separation and purification by flash chromatographic;
(2) the said signal generator is synthesized according to the following procedure:
CGGDEVD-(LS)_{N}-(Cy7-α-Lys)₁₀-NH₂
The process of the solid phase synthesis:
(a) Add the first amino acid to the Fmoc-Rink Amide TentaGel resin according to claim 12;
(b) place the following amino acids (each of the amino acids is 1 mmol) on the amino acid orbit of the ABI 433A automatic synthesizer with the following order:
(N-terminal) [Dde-Lys(Fmoc)-OH]₁₀ (C-terminal); proceed the automatic solid phase synthesis by machine; then treat the resin with 20% piperdine solution to deprotect, add 10 equivalents of compound prepared in step (1) compared to the active point of the resin, and 10 equivalents of DIPEA, react 2-5 hours at room temperature, then wash the resin, and continue to add the following amino acid by machine (start form deprotection):
(N-terminal) Fmoc-Cys(Trt)-OH, Fmoc-Gly-OH, Fmoc-Gly-OH, Fmoc-Asp(OtBu)-OH, Fmoc-Glu(OtBu)-OH, Fmoc-Val-OH, Fmoc-Asp(OtBu)-OH (C-terminal);
After the solid phase automatic synthesis finished, treat the resin with 2% Hydrazine solution to deprotect the Dde group in the Dde-Lys(Fmoc)-OH; add the 30 mmol Cy7-NHS produced by the GE Healthcare company into the above resin, after the reaction for 1 hour, wash the resin successively with DMF/DCM; the sequential treat is the same as that in claim 18;
(3) the said —NH₂ on the nano particles is transformed into maleimide by SMCC or Sulfo-SMCC, and then removing the excess SMCC or Sulfo-SMCC;
(4) restore the disulfide bond at its own hinge of the complete antibody molecules by excess dithiothreitol (DTT) to produce —SH functional group, and then removing the excess DTT;
(5) The product of step (4) is added to the product of step (3), coupling reaction between the —SH functional group produced in step (4) and the maleimide on the nano particles produced in step (3) occur in the buffer with pH 6-8, the molar ratio of the product of step (4) and the product of step (3) is: X:1 (X=2-20), stirred for 10-120 minutes; the product (previously dissolved in deionized water) of step (2) is then added, the molar ratio of the product of step (2) and the product of step (3) is: Y:1 (Y=2-100), and stirred for 10-300 minutes, and finally add an excess hydrotropic compound containing maleimide functional group, such as PEG-Maleimide, Sulfo-Maleimide, slight stirred for 10-300 minutes at 2-8° C.;
(6) remove the free antibody in the product of step (4), free product of step (1), the excess hydrotropic compound containing maleimide functional group, and the other residues of small molecule compounds, to obtain the target product: Antibodies—carrier—p1-(AA)ₙ-(LS)_{N}—X(F)-p2 system, and then add stabilizing agent into the target product, store the product at low temperature.

20. An kit (package) of the signal generator according to claim 1, including the usage of biological enzyme and the corresponding (AA)ₙ listed in the table 1 and table 2.
